(19) United States
(12) Patent Application Publication
(10) Pub. No.: US 2024/0226283 A9
(43) Pub. Date: Jul. 11, 2024

(54) VACCINATION OF HEMATOPOIETIC STEM CELL DONORS WITH CYTOMEGALOVIRUS TRIPLEX COMPOSITION

(71) Applicant: CITY OF HOPE, Duarte, CA (US)

(72) Inventors: Don J. DIAMOND, Duarte, CA (US); Corinna LA ROSA, Duarte, CA (US)

(73) Assignee: CITY OF HOPE, Duarte, CA (US)

(21) Appl. No.: 18/032,052

(22) PCT Filed: Oct. 15, 2021
(86) PCT No.: PCT/US21/55220

§ 371 Date: Apr. 14, 2023

Related U.S. Application Data
(60) Provisional application No. 63/092,741, filed on Oct. 16, 2020.

(51) Int. Cl. A61K 39/245; A61K 39/295; C12N 15/86

(52) U.S. Cl. CPC A61K 39/245; A61K 39/295; C12N 15/86; A61K 2039/5256

(57) ABSTRACT

Disclosed is a method of treating or preventing a CMV infection in a recipient of a hematopoietic cell transplant (HCT). The method entails administering an effective amount of a CMV Triplex vaccine composition to a donor and/or recipient of the hematopoietic cells.

### PRIORITY CLAIM

This application claims the benefit of U.S. Provisional Patent Application No. 63/092,741, filed Oct. 16, 2020, which is incorporated by reference herein in its entirety, including drawings.

### BACKGROUND

Despite modern therapeutics, cytomegalovirus (CMV) reactivation remains an important cause of morbidity and mortality post-hematopoietic cell transplant (HCT) (Teira et al., Blood 127(20): 2427-2438 (2016)). Letermovir (PREVYMIS™) demonstrated efficacy in preventing clinically significant CMV infections (Marty et al., N. Engl. J. Med. 377: 2433-2444 (2017)); however, when dosing ceased on day 100 post-HCT, the reactivation rate rebounded with evidence that CMV-specific immunity was impaired. This speculation was recently confirmed by investigators at the Fred Hutchinson Cancer Research Center who demonstrated that letermovir causes depression of CMV-specific immunity in the post-HCT setting (Zamora et al, Blood 138 (1):34-43 (2021)). This report proves that an alternative approach to control CMV reactivation is necessary, since letermovir can suppress the essential immune responses that will lead to long term resolution of CMV complications. Furthermore, there are reports of letermovir resistance, breakthrough viremia and CMV disease (Frietsch et al., Mediterr. J. Hematol. Infect. Dis. 11(1): e2019001 (2019); Knoll et al., Bone Marrow Transplant 54 (6): 911-912 (2019)).

Accordingly, there is an unmet need in the art for an alternative, immediately available, faster-acting, and safer approach to develop an effective prophylactic to protect an HCT recipient from CMV infection. The disclosed technology satisfies the need in the art.

### SUMMARY

This disclosure is directed to a method of eliciting or modifying an immune response and clinical protection against CMV infection in a subject who receives a hematopoietic cell transplant (HCT) by administering a vaccine composition to an HCT donor. The vaccine composition comprises an immunologically effective amount of a recombinant modified vaccinia Ankara (rMVA) vector inserted with two or more nucleic acid sequences encoding two or more CMV antigens or antigenic portions thereof. In some embodiments, the CMV antigens or antigenic fragments thereof include IE1 or IE1 exon 4 (IE1/e4), IE2 or IE2 exon 5 (IE2/e5), IEfusion (e.g. fusion of IE1/e4 and IE2/e5), and pp65. In various embodiments, pp65 can be co-expressed with IE1 or IE1/e4, IE2 or IE2/e5, or IEfusion. In some embodiments, two or more nucleic acid sequences are operably linked to and under the control of a single promoter, such as the mH5 promoter. In other embodiments, each nucleic acid sequence is operably linked to and under the control of a separate mH5 promoter. Additionally, other poxvirus promoters can be used and the use of an mH5 promoter is not required. In some embodiments, the two or more nucleic acid sequences are inserted in the same insertion site. In some embodiments, the two or more nucleic acid sequences are inserted in different insertion sites. The insertion sites include, e.g., 044L/045L, IGR3, G1L/18R, and Del 3. In some embodiments, the nucleic acid encoding the CMV antigen is codon optimized, e.g., to remove consecutive cytosines or guanines while expressing the same amino acids. In some embodiments, the donor, the recipient, or both are mammal, such as human. In some embodiments, the HCT donor receives one, two, or three doses of the vaccine composition. In some embodiments, the HCT recipient is also administered with one or more doses of the vaccine composition after HCT. In some embodiments, the vaccine composition is administered by intramuscular administration, intradermal administration, subcutaneous, administration, intravenous administration, intranasal administration, or intraperitoneal administration. In some embodiments, the HCT donor is administered with one or more doses of a CMV Triplex vaccine composition 10-60 days prior to the start of stem cell mobilization. In some embodiments, the recipient undergoes HCT within 9 weeks of the donor's vaccination. In some embodiments, the recipient is administered with one or more doses (e.g., a single dose, two doses, or three doses) of a CMV Triplex vaccine composition between day 28 and day 100 post-transplant. If needed, the recipient can receive a CMV Triplex vaccine composition beyond day 100 post-transplant. In some embodiments, the HCT is a human leukocyte antigen (HLA)-matched transplant. In some embodiments, the HCT is a haploidentical (HLA half-matched) transplant. In some embodiments, the HCT is a mismatched transplant.

In a related aspect, this disclosure is directed to a method of treating or preventing a subject who receives a hematopoietic cell transplant from CMV infection by administering a vaccine composition to an HCT donor. The vaccine composition comprises an immunologically effective amount of a recombinant modified vaccinia Ankara (rMVA) vector inserted with two or more nucleic acid sequences encoding two or more CMV antigens or antigenic portions thereof. In some embodiments, the CMV antigens or antigenic fragments thereof include IE1 or IE1 exon 4 (IE1/e4), IE2 or IE2 exon 5 (IE2/e5), IEfusion (e.g. fusion of IE1/e4 and IE2/e5), and pp65. In various embodiments, pp65 can be co-expressed with IE1 or IE1/e4, IE2 or IE2/e5, or IEfusion. In some embodiments, two or more nucleic acid sequences are operably linked to and under the control of a single promoter, such as the mH5 promoter. In other embodiments, each nucleic acid sequence is operably linked to and under the control of a separate mH5 promoter. Additionally, other poxvirus promoters can be used and the use of an mH5 promoter is not required. In some embodiments, the two or more nucleic acid sequences are inserted in the same insertion site. In some embodiments, the two or more nucleic acid sequences are inserted in different insertion sites. The insertion sites include, e.g., 044L/045L, IGR3, G1L/18R, and Del 3. In some embodiments, the nucleic acid encoding the CMV antigen is codon optimized, e.g., to remove consecutive cytosines or guanines while expressing the same amino acids. In some embodiments, the donor, the recipient, or both are mammal, such as human. In some embodiments, the HCT donor receives one, two, or three doses of the vaccine composition. In some embodiments, the HCT recipient is also administered with one or more doses of the vaccine composition after HCT. In some embodiments, the vaccine composition is administered by intramuscular administration, intradermal administration, subcutaneous, administration, intravenous administration, intranasal administration, or intraperitoneal administration. In some embodiments, the HCT donor is administered with one or more doses of a CMV Triplex vaccine composition 10-60 days prior to the start of stem cell mobilization. In some embodiments, the recipient undergoes HCT within 9 weeks of the donor's vaccination. In some embodiments, the recipient is administered with one or more doses (e.g., a single dose, two doses, or three doses) of a CMV Triplex vaccine composition between day 28 and day 100 post-transplant. In some embodiments, the HCT is an HLA-matched transplant. In some embodiments, the HCT is a haploidentical (HLA half-matched) transplant. In some embodiments, the HCT is a mismatched transplant.

### BRIEF DESCRIPTION OF THE DRAWINGS

FIG. 1 illustrates a schematic representation of Triplex vaccine, mH5-pp65-IEFusion-MVA (marker gene free). FL1 and FL2 are flanking (FL) DNA of deletions II and III. TK, thymidine kinase gene of MVA. Arrows show direction of transcription.

FIG. 2 illustrates donor only Triplex vaccination scheme.

FIG. 3 illustrates donor-and-recipient Triplex vaccination scheme.

FIG. 4 shows measurement of T cell responses in 24 healthy research subjects pre and post vaccination (d14-d360). Using the CD137 T cell activation assay after incubation of PBMC with 138 peptides comprising the pp65 antigen (Pepmix, JPT) from Longitudinal blood draws from each of 8 vaccine recipients. Arrow shows injection.

FIG. 5 shows the comparison of CMV-specific CD8 T cell levels in 12 patients receiving an HCT from a matched related donor (MRD) vaccinated with a CMV Triplex vaccine composition versus a control cohort of MRD HCT recipients (N=41).

FIG. 6 shows the longitudinal immune profiles of unique patient number (UPN) 1, 3, 9, and 12 CMV-specific T cells. UPN 1 (top left), UPN 3 (top right), UPN 9 (bottom left), and UPN 12 (bottom right).

### DETAILED DESCRIPTION

The technology disclosed herein entails supplying a vulnerable HCT recipient with immune-mediated protection by transfer of immune cells from the donor thereby to achieve early protection against CMV viremia in the transplant field. Accordingly, disclosed herein is an innovative approach to protect HCT recipients from CMV viremia early after transplant by vaccinating the donor prior to stem cell harvest before the recipient can achieve adequate immune responses with a CMV Triplex vaccine composition. In some embodiments, if there is a delay in the transplant procedure, the stem cell product obtained from the previously vaccinated donor can be routinely frozen, thawed in time for the rescheduled transplant, and the CMV-specific T cells are still present and acting to protect the recipient from the complications of CMV reactivation. The Triplex vaccine compositions used for vaccinating the HCT donors, as well as methods of producing these Triplex vaccine compositions, are disclosed in the inventor's prior patent publications such as U.S. Pat. Nos. 8,580,276, 9,675,689, 10,603,375, and WO 2019/217922, the contents of which are incorporated by reference in their entireties as well as in the prior publications (La Rosa et al., Blood 129(1): 114-125 (2017); Aldoss et al., Ann. Intern. Med. 172(5): 306-316 (2020)). U.S. Pat. No. 9,675,689 disclosing a first generation CMV Triplex vaccine composition and WO 2019/217922 disclosing a second generation CMV Triplex vaccine composition are submitted herewith and constitute part of the specification. These CMV Triplex vaccine compositions can be used to vaccinate the HCT donor and/or recipient as disclosed herein.

As used herein, a CMV “Triplex” vaccine composition refers to a recombinant MVA (rMVA) comprising one or more nucleotide sequences encoding one or more CMV antigens or an immunogenic fragment thereof, such as an Immediate-Early Gene-1 (IE-1) or exon 4 of IE1, an Immediate-Early Gene-2 (IE-2) or exon 5 of IE2, and pp65. In certain embodiments, two or more CMV antigens or immunogenic fragments thereof can form a fusion. The first generation Triplex vaccine compositions are disclosed in U.S. Pat. No. 9,675,689. The second generation Triplex vaccine compositions have improved genetic stability over serial passages compared to the first generation Triplex vaccine compositions, as disclosed in WO 2019217922. Other poxvirus platforms such as sMVA (e.g., disclosed in PCT Publication No. WO 2021/158565), or other poxvirus vehicles well known to those skilled in the art may be used to deliver the Triplex CMV antigens including pp65, IE1, and IE2. Other antigen delivery mechanisms such as adenovirus (Ad26 and its derivatives, Chadox and its derivatives, or other platforms such as attenuated measles, vesicular stomatitis virus, LCMV (M. Schwendinger et al, J. of Infectious Diseases, Accession #32313928, doi: 10.1093/infdis/jiaa121), or more recent technologies such as the mRNA-based vaccine platform that have been investigated in the CMV field (Plotkin et al, J. of Infectious Disease 221 (Suppl 1): S113-S122, 2020 PMC7057802).

Live viral vaccination aims to induce helper and cytotoxic immunity and hence a durable memory response. Plotkin et al. developed a therapeutic vaccine, the attenuated Towne strain, in the 1970's. However, concerns regarding live CMV have minimized its applicability. Latter attempts include ALVAC expressing gB (UL55), which failed to elicit significant antibody levels in CMV-negatives, and ALVAC-UL83 which stimulated robust cellular immunity in CMV-negatives equivalent to natural CMV positives. Further studies with ALVAC-UL55 and purified soluble UL55 protein demonstrated minimal efficacy. AlphaVax™ expressing UL83, UL123 and UL55 was promising in healthy adults, but is unsuitable for HCT recipients since it can propagate in humans. Despite promising animal data, TransVax™, a DNA vaccine expressing either UL55 or UL83 induced only weak responses in humans.

CMVPepVax, derived from the CMV-UL83 antigen, was safe and elicited vaccine driven immune responses when tested in healthy adults (NCT00722839). Subsequently CMVPepVax was found to be safe in HCT recipients (NCT01588015) when injected on day 28 and day 56 post-HCT, with reduced CMV reactivation and no increase in acute GVHD. However, the application of CMVpepVax is restricted to the HLA A*0201 population, who comprise only ˜30-40% of the HCT population. The Triplex vaccine being investigated in this protocol has no HLA restriction. It shows greater immunogenicity than DNA vaccines, and since it expresses whole CMV proteins, has broader recognition and greater applicability for HCT recipients than CMVPepVax.

Construction, expression and function of the Triplex vaccine compositions are disclosed in detail in the appendices and briefly summarized below.

Choice of antigens: Since they are targets for cell mediated immune responses, UL83, UL122 and UL123 have been selected as vaccine antigens. UL83 is the most immunogenic CMV structural protein, although UL123 may be comparable. All three are immunodominant, and combined recognition should occur in over 95% of the population. An association of cellular immunity to UL83 and UL123 with recovery from CMV-retinitis in AIDS patients has been reported. Furthermore, T cells specific for these antigens accumulate in individuals with CMV reactivation episodes. Although there is a strong humoral response to Triplex, there is no evidence that this neutralizes CMV. The majority of the CMV-neutralizing antibody response has been localized to the gB (UL55) and UL128 gene products [99, 121-124]. Evidence that a humoral response protects HCT recipients against CMV is lacking, hence gB has been omitted from this vaccine. The Triplex vaccine focuses on the cell mediated response associated with disease protection in HCT recipients.

Functional modification of CMV genes incorporated into MVA: the regulatory activity of the UL123 protein includes trans-activating properties on various cellular promoters. Consequently 85 aa comprising coding exons 2 and 3 have been deleted. Deletion of the two coding exons results in a cytoplasmic, 406-aa protein with minimal transactivation activity. Most known CTL epitopes from UL123 are found in exon 4, including the HLA A*0201-restricted CTL epitopes. Exon5 of UL122 was fused in frame to exon4 of UL123 without modification.

Host cells for Triplex generation: MVA was derived by serial transfer (570 passages) of the parental Ankara strain through chicken embryo CEF to derive a safe alternative to the smallpox vaccine. Its adaptation to CEF resulted in several genomic deletions. These adaptations allow MVA to freely propagate in CEF to titers exceeding 10E10 pfu/mL, whereas standard mammalian cell lines such as CV-1 are non-permissive for propagation. For the pre-clinical studies conducted under GLP, specific pathogen free CEF, from Charles River-SPAFAS were used. Triplex vector was constructed using the pZWIIA plasmid and insertion of foreign genes by homologous recombination. The modified H5 (mH5) promoter ensures sufficient protein expression for manufacture of a stable virus, providing a powerful boost to transgene expression without causing genomic instability.

The rMVA expressing immunodominant CMV antigens including pp65, IE1 and IE2 were used to immunize matched related donors of CMV seropositive recipients. No adverse events possibly or likely related to the vaccine were reported. Early post-HCT development of robust and long-lasting frequency of pp65-, IE1- and IE2-specific CD4 and CD8 T cells was observed in all recipients. Memory profiles of CMV specific T cells had marked prevalence of effector memory phenotype early post-HCT, which persisted.

Thus, this disclosure relates to transfer of protective CMV-specific immunity in recipients receiving HCT from a donor vaccinated with an immunologically effective amount of a CMV Triplex vaccine composition. In some embodiments, the HCT donor and recipient are HLA matched. In some embodiments, the HCT donor is haploidentical (HLA half-matched) to the HCT recipient. In some embodiments, the HCT donor is unrelated to the HCT recipient. In some embodiments, only the HCT donor is vaccinated with the CMV Triplex vaccine composition such that the HCT recipient acquires immunity to CMV infection from the donor. In some embodiments, both the HCT donor and the HCT recipient are vaccinated with the CMV Triplex vaccine composition.

The superior preliminary clinical and immunological results indicate that this novel immune therapeutic strategy provides a safer alternative to antivirals, including letermovir prophylaxis and can be applied to higher CMV risk patients, such as haploidentical HCT recipients. This approach demonstrates the benefits of safety and efficacy by vaccinating an HCT donor, to elicit early CMV protective immune recovery in CMV seropositive HCT recipients, who are at high risk of developing CMV serious complication after HCT.

An “immunologically effective amount” as used herein means an amount that is both safe to a donor or recipient subject (animal or human) to be immunized and sufficient to improve the immunity of the recipient subject to CMV infection. The immunologically effective amount can vary and can be determined by means of known art through routine trials. For example, one or more doses of the CMV Triplex vaccine can be administered to the HCT donor or recipient. The immunologically effective amount can vary from about 1×10⁶ pfu to about 1×10⁹ pfu in a volume from about 0.1 mL to about 1.0-2.0 mL of suspended vaccine. For example, an immunologically effective amount is about 1×10⁶ pfu, about 5×10⁶ pfu, about 1×10⁷ pfu, about 5×10⁷ pfu, about 1×10⁸ pfu, about 5×10⁸ pfu, or about 1×10⁹ pfu.

The CMV Triplex vaccine, a recombinant modified vaccinia Ankara expressing immunodominant CMV antigens (pp65, IE1 and IE2) or immunogenic fragments thereof, is being evaluated by immunizing donors of CMV seropositive recipients. The CMV Triplex vaccine, developed to elicit and enhance protective CMV-specific T cells, is a promising vaccine that demonstrated excellent tolerability and immunogenicity in healthy adults, and in HCT CMV seropositive recipients, who had a 50% reduction in CMV reactivation requiring preemptive antiviral therapy, and significantly enhanced CMV-specific immune responses.

The working examples demonstrate the transfer of CMV-specific immunity in recipients receiving an HCT from a matched related donor who has been vaccinated with the CMV Triplex vaccine.

This is the first study to assess the possible benefit of vaccinating MRD, to elicit early CMV immune recovery in CMV seropositive HCT recipients, at high risk for developing CMV serious complication after HCT. The approach is feasible and highly tolerable, with promising evidence of efficacy, indicating that Triplex vaccination of the MRD confers early protective anti-CMV immunity to the HCT recipient. Clinical and immunological outcomes from the ongoing study indicate that this novel immune therapeutic strategy provides a safer alternative to antivirals, including letermovir prophylaxis and may be used in higher CMV risk patients, such as haploidentical HCT recipients.

From the foregoing, it will be appreciated that specific embodiments of the invention have been described herein for purposes of illustration, but that various modifications may be made without deviating from the scope of the invention. The examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

### Example 1. Development of Triplex

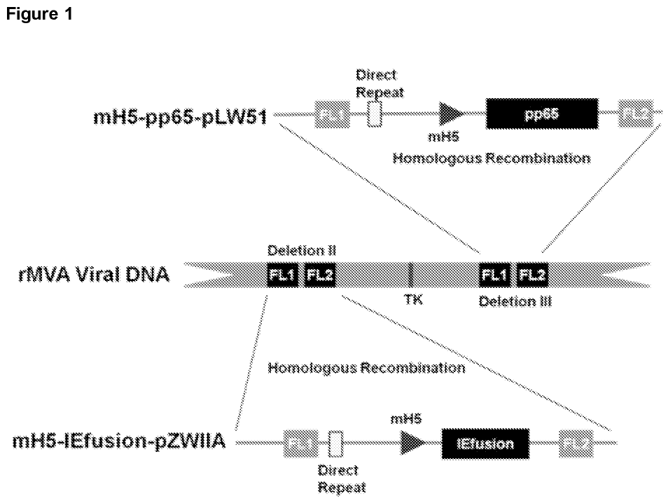

An attenuated multiple-antigen recombinant MVA (Triplex) with genes encoding 3 immunodominant CMV proteins: pp65 (UL83), IE1-exon4 (UL123), and IE2-exon5 (UL122) with assistance from the NCI-NEXT program (Wang, Z., et al. Vaccine 28: 1547-1557 (2010)). Triplex was constructed using an MVA viral backbone and 2 recombinant shuttle vectors, mH5-pp65-pLW51 and mH5-IEfusion-pZWIIA containing all 3 CMV genes within 2 transgenes that were inserted into the viral genome using homologous recombination (FIG. 1). Parental wild type (wt) MVA virus (MVA 572.FHE-22.02.1974) used to construct Triplex was provided by Dr. Bernard Moss, (Laboratory of Viral Disease, NIAID, NIH, Bethesda, MD). One transgene is composed of a fusion protein of 2 CMV antigens, immediate-early 1 (IE1, UL123-exon4) and immediate early 2 (IE2, UL122-exon5), and was inserted in the MVA deletion-II locus. The second transgene contains another CMV antigen, pp65 (UL83), and was inserted in the MVA deletion-Ill locus. The transcription of each transgene is under the control of separate mH5 promoters120. The preclinical safety and immunogenicity was established using HLA transgenic mice (A2, B7, A1, and A11) and PBMC from CMV-P healthy volunteers (HV) and HCT-R76,119. Triplex vaccine was manufactured at the Center for Biomedicine and Genetics (CBG) at COH.

### Example 2. Triplex Vaccination Schema

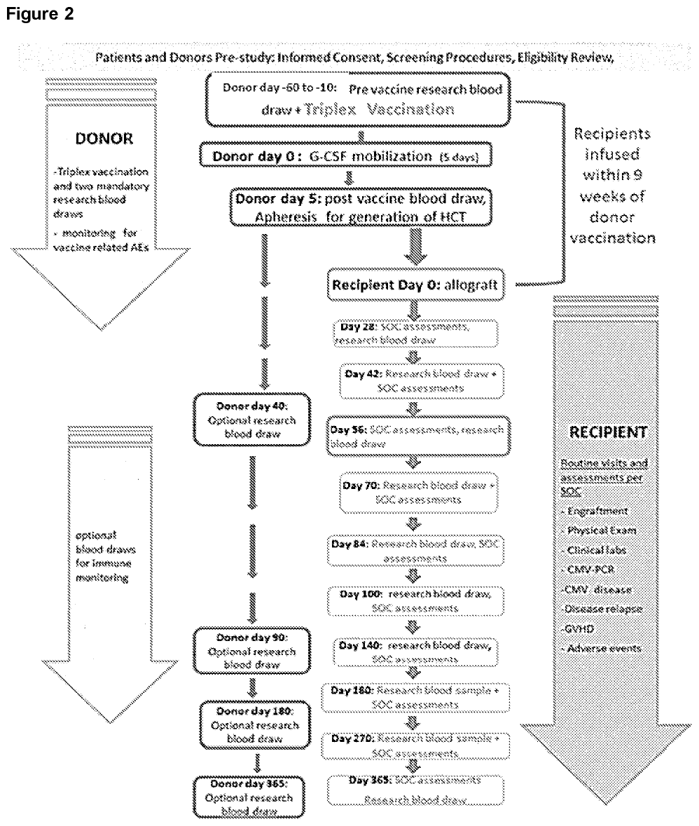

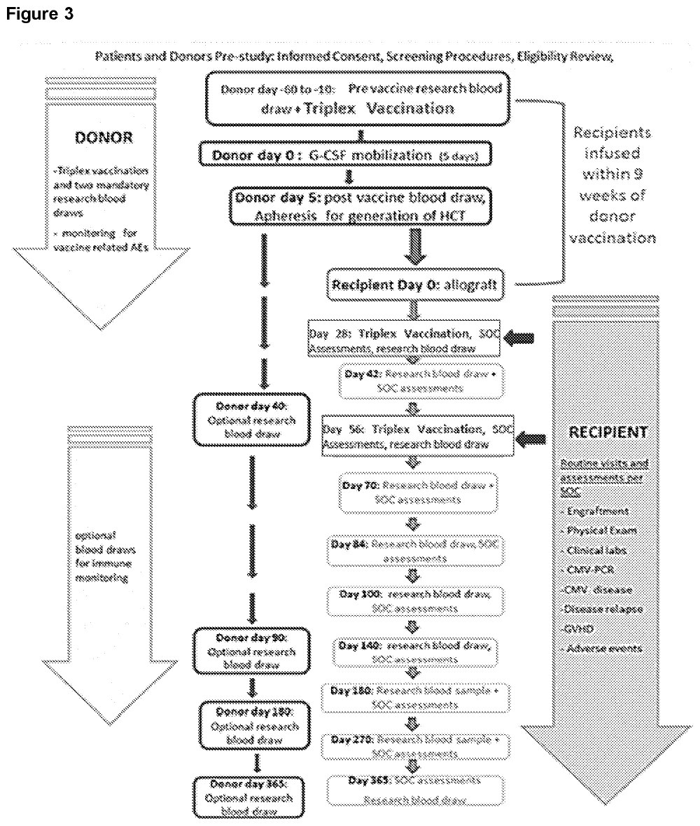

CMV+ adults about to undergo 8/8 high resolution HLA donor allele matching or 3/6 HLA donor allele (haploidentical) matching hematopoietic stem cell transplant (HCT) for the treatment of hematologic malignancy. FIGS. 2 and 3 illustrate the donor-only vaccination and donor-and-recipient vaccination, respectively.

The schedule of procedures is illustrated as follows. Donor: Day −60 to −10, Triplex vaccination; Day −5 to −1, GCSF mobilization of vaccinated donor; Day −1 to 0: PBMC harvest and preparation of HCT graft. Recipient: Day 0, HCT PBSC transplant; Day 28, Triplex vaccination (for donor-and-recipient vaccination scheme only); Day 56: Triplex vaccination (for donor-and-recipient vaccination scheme only). Donor days are measures from the first day of GCSF administration. Recipient days are measured from the day of transplant.

### Example 3. Triplex Vaccine Administration in Healthy Adults

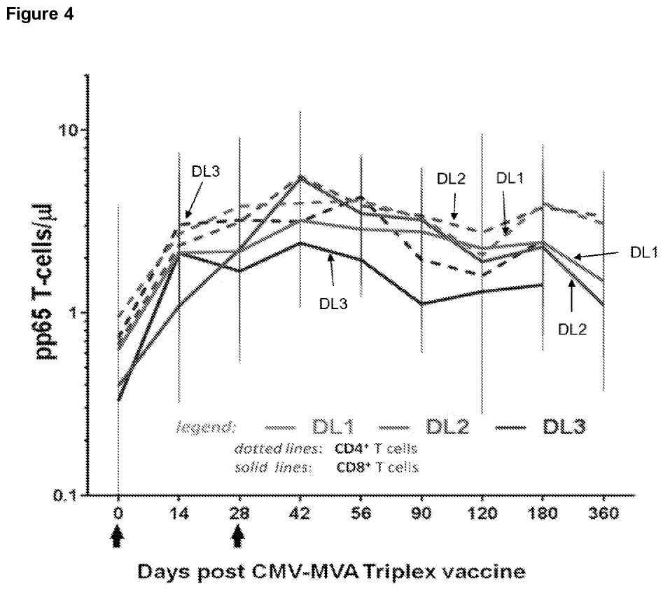

Measuring viral persistence, maximum tolerated dose (MTD) and immunogenicity of Triplex in healthy volunteers (HV) was required by the FDA prior to treatment of HCT recipients. In the Phase I trial (NCT01941056), these endpoints were evaluated in 24 HV (age: 18-60), with or without prior immunity to CMV and vaccinia. Three escalating dose levels (DL) were administered intramuscularly (DL1=10×E7; DL2=5×10E7; DL3=5×10E8 pfu/dose) in 8 subjects/DL, with a booster injection 28 d later. Subjects were followed for 1 year. All 24 planned HV were enrolled, vaccinated and completed 12 months of planned follow-up. All vaccinations were well-tolerated, with no SAE or DLT. Immunogenicity of the vaccine was evaluated by measuring activation of T cells harvested from vaccinees and stimulated with full-length pp65, IE-1 and IE2 overlapping peptide libraries, or quantification of CMV-specific T-cells with HLA multimers. Triplex induced robust expansion of pp65, IE1 and IE2-specific CD8 and CD4 T-cells in vaccinated CMV positives, at each DL, shown in FIG. 4. CMV-specific T-cells with common HLA alleles and corresponding CMV-CTL epitopes were identified. Statistical analysis indicated that post-vaccination levels of pp65-, IE1- or IE2-specific CD8 and CD4 T-cells were significantly increased (p-values ranging from 3×10⁻⁵ to 0.025). Importantly, robust immunity was detected in CMV negatives and in subjects who had received smallpox vaccinations. Elevated frequencies of CMV-specific CD4 and CD8 T cells for all 3 antigens plateau after day 56, and in some cases remain elevated for one year. Furthermore, naïve T cells dropped during the vaccination phase and terminal effector-memory T cells rose, suggesting effective recognition by CMV-specific T cells followed by evolution to a more mature effector-memory phenotype. PCR assessment of circulating vector showed minimal residual MVA DNA [10-30 gc/mL] post-injection in 2 vaccinees in the DL3 cohort, which disappeared within 3 months. This demonstrates that CMV+ patients receiving allografts from CMV+ or CMV− donors would generate protective CMV-specific immunity after vaccination with Triplex.

### Example 4. Assessment of the Immune Response in HCT Recipients

GMP Triplex Production: CEF cells were seeded at a density of 4.9×10⁴ cells/cm2 in T225 flasks containing complete VP-SFM Media (Life Sciences) and incubated for ˜96 hours at 37° C., 95% humidity and 5% CO₂. The total number of viable cells were determined from one flask using trypan blue exclusion. The media were replaced for the control flasks and the remaining flasks infected at an MOI of 0.02 using the Master Viral Seed Stock (MVSS), Batch #0825-181-0001. Each flask, containing approximately 9.2×10⁶ cells, was infected with 1.8×10⁵ pfu of MVSS, with Cytopathic effect observed ˜48 hours post-infection. About 4L per sub-batch of the harvested crude cell suspension was collected and ˜280 mL of sample from each sub-batch collected for QC testing. The remaining volume was centrifuged for 15′ at 1500 rpm (491×g) using a Sorvall RT-7 centrifuge. Cell pellets were collected and frozen in a −80° C. freezer for up to 96 hours prior to purification. Purification of Triplex from each sub-batch pellet was performed on separate days. Virus-infected pellets were thawed, resuspended in 84 mL of 10 mMTris-HCL, pH 9.0 and homogenized on ice, using 100 a 40 mL Dounce Tissue grinder. The homogenized cell suspension was sonicated twice for 30″ (using one second pulse cycles), being placed on ice between each round of sonication. The homogenate was then centrifuged for 10′ at 1600 rpm (558×g) using a Sorvall RT-7 centrifuge to remove cell debris. A 30′-45′ Benzonase® incubation step, using 500 units of enzyme per mL of supernatant was performed at 37° C. The virus suspension was then layered in ultracentrifuge tubes containing 15 mL of 36% sucrose and spun at 32,000×g using a Beckman Optima L90K for 80 minutes at 4°C. The effluent was removed and subsequent washes of the pellet performed. The wash step included reconstitution of the pellet in 1mM Tris-HCl, pH 9.0 and ultracentrifugation for 60′ at 4° C., 32,000×g. After the second and final wash, the effluent was removed and the viral pellets reconstituted in 7.5% Lactose/PBS. Each sub-batch was tested for sterility. Clinical lots were prepared by thawing the bulk product containers at room temperature and pooling four purified sub-batches. The prepared pooled bulk was diluted to achieve a final concentration of 5.0-6.0×10⁸ pfu/mL in 7.5% lactose/PBS. The Triplex vaccine was supplied frozen at approximately 9.1×10⁸ pfu/mL/vial in the formulation buffer of PBS, 7.5% lactose.

After obtaining informed consent, 18-75-year old HCT recipients (CMV seropositive) with matched related donors (MRDs) underwent T cell replete HCT. Donors received one injection of the first generation Triplex vaccine composition 10-60 days prior to start of stem cell mobilization, and recipients underwent HCT within 9 weeks of donor vaccination. The Triplex vaccine composition had a concentration of 5.1×10E8 pfu/ml in PBS containing 7.5% lactose, as described in La Rosa et al., Blood 129(1): 114-125 (2017); and Aldoss et al., Ann. Intern. Med. 172(5): 306-316 (2020) or concentrations greater than 5.1×10E8 pfu in newer clinical lots as the initial clinical lot was used up. As per standard of care, the vaccinated donors were mobilized with granulocyte colony-stimulating factor prior to apheresis. The study had a target of 18 HCT donor/recipient pairs. All HCT recipients are followed until day 365 post-HCT for safety, virologic and immunologic assessment. All donors are monitored for vaccine induced adverse events. Antiviral treatment for viremia is considered a failure of donor vaccination to provide protection to the recipient.

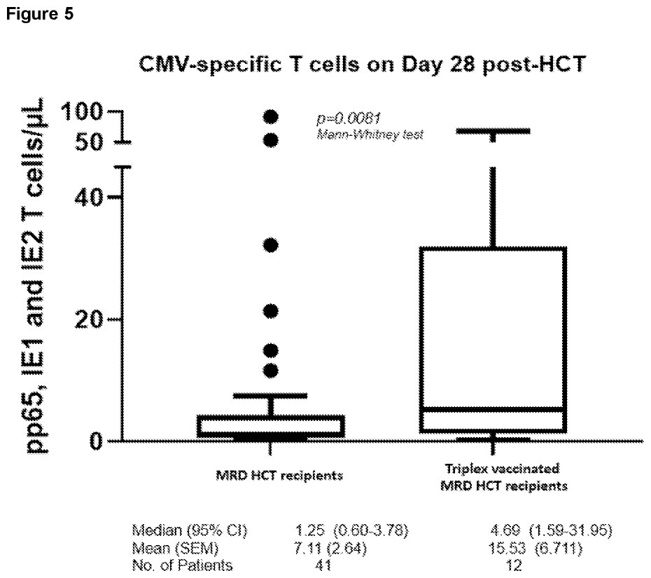

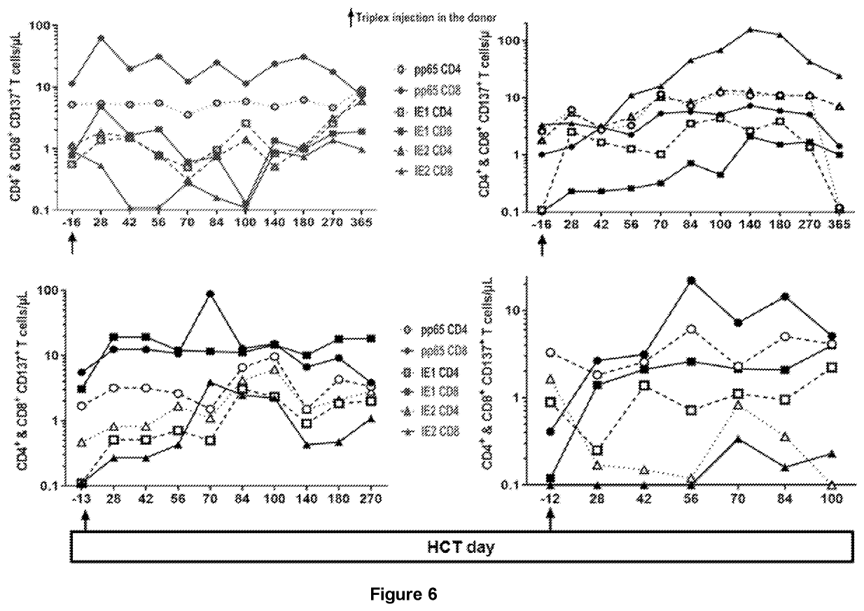

Eighteen MRD/recipients have been enrolled into the study and seventeen MRDs haven been safely vaccinated with the first generation Triplex vaccine composition, and infused with mobilized peripheral blood stem cells with T cells by volume approximately 10-25% to their recipients. No adverse events possibly or likely related to the vaccine were reported. Preemptive antiviral treatment was administered to one recipient (UPN 11) whose CMV seronegative donor had a low response early post Triplex vaccine and two more recipients (UPN 15 and UPN 17) who had a complicated post-transplant course, causing marked lymphopenia. Nine donor/recipient pairs have reached study end, and immune monitoring has been completed, whereas four donor/recipient pair have been evaluated immunologically to day 100 post-HCT. The flow cytometry panel of cellular immune assays included measuring concentrations of CMV-specific T cells expressing the 4-1BB (CD137) functional activation marker and assessing the memory phenotype profiles (performed as detailed in La Rosa et al., Blood 129(1): 114-125 (2017). Peripheral blood mononuclear cells (PBMC) were stimulated for 24 hours with either pp65, IE1 and IE2 overlapping 15mer peptide libraries and then stained with fluorescently tagged antibodies against CD137, CD3, CD8, CD4, combined with CD28 and CD45RA memory markers by using a Beckman-Coulter Gallios cytometer with Kaluza software. Early post-HCT development of robust and long-lasting frequency of pp65-, IE1- and IE2-specific CD4 and CD8 T cells was observed in the recipients (FIG. 6). Memory profiles of CMV specific T cells had marked prevalence of effector memory phenotype early post-HCT, which persisted over time. Interestingly, on day 28 post-HCT, pp65-specific CD8 T cell levels were significantly higher in the 12 recipients who received an HCT from an MRD vaccinated with the first generation Triplex vaccine composition compared to a control cohort of consecutively enrolled MRD HCT recipients (FIG. 5).

1. A method of eliciting or modifying an immune response and clinical protection against CMV infection in a subject who receives a hematopoietic cell transplant (HCT), comprising administering a vaccine composition to a donor of the hematopoietic cell, wherein the vaccine composition comprises an immunologically effective amount of a recombinant modified vaccinia Ankara (rMVA) vector inserted with two or more nucleic acid sequences encoding two or more CMV antigens or antigenic portions thereof.

2. The method of claim 1, wherein the CMV antigens or antigenic fragments thereof include IE1 or IE1 exon 4 (IE1/e4), IE2 or IE2 exon 5 (IE2/e5), IEfusion of IE1/e4 and IE2/e5, and pp65.

3. The method of claim 2, wherein pp65 is co-expressed with IE1 or IE1/e4, IE2 or IE2/e5, or IEfusion.

4. The method of any one of claims 1-3, wherein two or more nucleic acid sequences are operably linked to and under the control of a single promoter.

5. The method of claim 4, wherein the promoter is the mH5 promoter.

6. The method of any one of claims 1-3, wherein each nucleic acid sequence is operably linked to and under the control of a separate promoter.

7. The method of any one of claims 1-6, wherein the donor is a human.

8. The method of any one of claims 1-7, wherein the recipient is a human.

9. The method of any one of claims 1-8, wherein the vaccine composition is administered to the donor by intramuscular administration, intradermal administration, subcutaneous, administration, intravenous administration, intranasal administration, or intraperitoneal administration.

10. The method of any one of claims 1-9, wherein the donor receives one, two, or three doses of the vaccine composition.

11. The method of any one of claims 1-10, wherein the donor receives a single dose of the vaccine composition 10-60 days prior to the start of stem cell mobilization.

12. The method of any one of claims 1-11, wherein the recipient undergoes HCT within 9 weeks of the donor's vaccination.

13. The method of any one of claims 1-12, wherein the recipient receives one or more doses of the vaccine composition after HCT.

14. The method of any one of claims 1-13, wherein the recipient receives one or more doses of the vaccine composition between day 28 and day 100 post-transplant or beyond day 100 post-transplant.

15. The method of any one of claims 1-14, wherein the HCT is HLA-matched.

16. The method of any one of claims 1-14, wherein the HCT is haploidentical or mismatched.

17. A method of treating or preventing a subject who receives a hematopoietic cell transplant (HCT) from CMV infection, comprising administering a vaccine composition to a donor of the hematopoietic cell, wherein the vaccine composition comprises an immunologically effective amount of a recombinant modified vaccinia Ankara (rMVA) vector inserted with two or more nucleic acid sequences encoding two or more CMV antigens or antigenic portions thereof.

18. The method of claim 17, wherein the CMV antigens or antigenic fragments thereof include IE1 or IE1 exon 4 (IE1/e4), IE2 or IE2 exon 5 (IE2/e5), IEfusion of IE1/e4 and IE2/e5, and pp65.

19. The method of claim 18, wherein pp65 is co-expressed with IE1 or IE1/e4, IE2 or IE2/e5, or IEfusion.

20. The method of any one of claims 17-19, wherein two or more nucleic acid sequences are operably linked to and under the control of a single promoter.

21. The method of claim 20, wherein the promoter is the mH5 promoter.

22. The method of any one of claims 17-19, wherein each nucleic acid sequence is operably linked to and under the control of a separate promoter.

23. The method of any one of claims 17-22, wherein the donor is a human.

24. The method of any one of claims 17-23, wherein the recipient is a human.

25. The method of any one of claims 17-24, wherein the vaccine composition is administered to the donor by intramuscular administration, intradermal administration, subcutaneous, administration, intravenous administration, intranasal administration, or intraperitoneal administration.

26. The method of any one of claims 17-25, wherein the donor receives one, two, or three doses of the vaccine composition.

27. The method of any one of claims 17-26, wherein the donor receives a single dose of the vaccine composition 10-60 days prior to the start of stem cell mobilization.

28. The method of any one of claims 17-27, wherein the recipient undergoes HCT within 9 weeks of the donor's vaccination.

29. The method of any one of claims 17-28, wherein the recipient receives one or more doses of the vaccine composition after HCT.

30. The method of any one of claims 17-29, wherein the recipient receives one or more doses of the vaccine composition between day 28 and day 100 post-transplant or beyond day 100 post-transplant.

31. The method of any one of claims 17-30, wherein the HCT is HLA-matched.

32. The method of any one of claims 17-30, wherein the HCT is haploidentical or mismatched.


SEQUENCE LISTING (46 sequences)

SEQ ID NO: 1 - DNA, 22 bp, Artificial Sequence
  pp65 forward primer
atcaaaccgggcaagatctcgc

SEQ ID NO: 2 - DNA, 22 bp, Artificial Sequence
  pp65 reverse primer
atcgtactgacgcagttccacg

SEQ ID NO: 3 - DNA, 20 bp, Artificial Sequence
  IE1 exon4 forward primer
ccatcgccgaggagtcagat

SEQ ID NO: 4 - DNA, 20 bp, Artificial Sequence
  IE1 exon4 reverse primer
agtgtcctcccgctcctcct

SEQ ID NO: 5 - DNA, 20 bp, Artificial Sequence
  IEfusion forward  primer
aagttgccccagaggaagag

SEQ ID NO: 6 - DNA, 20 bp, Artificial Sequence
  IEfusion reverse primer
ctgctaacgctgcaagagtg

SEQ ID NO: 7 - DNA, 21 bp, Artificial Sequence
  TK forward primer
tgtgagcgtatggcaaacgaa

SEQ ID NO: 8 - DNA, 28 bp, Artificial Sequence
  TK reverse primer
tcgatgtaacactttctacacaccgatt

SEQ ID NO: 9 - DNA, 9388 bp, Artificial Sequence
  mH5-IEfusion-pZWIIA (GUS) plasmid DNA sequence
cctcctgaaaaactggaatttaatacaccatttgtgttcatcatcagacatgatattactggatttatattgtttatgggtaaggtagaatctccttaatatgggtacggtgtaaggaatcattattttatttatattgatgggtacgtgaaatctgaattttcttaataaatattatttttattaaatgtgtatatgttgttttgcgatagccatgtatctactaatcagatctattagagatattattaattctggtgcaatatgacaaaaattatacactaattagcgtctcgtttcagacatggatctgtcacgaattaatacttggaagtctaagcagctgaaaagctttctctctagcaaagatgcatttaaggcggatgtccatggacatagtgccttgtattatgcaatagctgataataacgtgcgtctagtatgtacgttgttgaacgctggagcattgaaaaatcttctagagaatgaatttccattacatcaggcagccacattggaagataccaaaatagtaaagattttggctattcagtggactggatgattcgaggtaccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctgggtaccaggcgcgcatttcattttgtttttttctatgctataaatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggcggcccgctcgagaaaaattgaaaataaatacaaaggttcttgagggttgtgttaaattgaaagcgagaaataatcataaataagccaccaccgtttaaacgccaccacaatggtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaaaataaaggcgcgccataaaaatttttatactagtgtaccgcggtcgaatcgatttaattaacgatgctagcattgtcgacggtggtggcgcggccgccagtgtgatggatatctgcagaattcggcttggggggctgcaggtggatgcgatcatgacgtcctctgcaatggataacaatgaacctaaagtactagaaatggtatatgatgctacaattttacccgaaggtagtagcatggattgtataaacagacacatcaatatgtgtatacaacgcacctatagttctagtataattgccatattggatagattcctaatgatgaacaaggatgaactaaataatacacagtgtcatataattaaagaatttatgacatacgaacaaatggcgattgaccattatggagaatatgtaaacgctattctatatcaaattcgtaaaagacctaatcaacatcacaccattaatctgtttaaaaaaataaaaagaacccggtatgacacttttaaagtggatcccgtagaattcgtaaaaaaagttatcggatttgtatctatcttgaacaaatataaaccggtttatagttacgtcctgtacgagaacgtcctgtacgatgagttcaaatgtttcattgactacgtggaaactaagtatttctaaaattaatgatgcattaatttttgtattgattctcaatcctaaaaactaaaatatgaataagtattaaacatagcggtgtactaattgatttaacataaaaaatagttgttaactaatcatgaggactctacttattagatatattctttggagaaatgacaacgatcaaaccgggcatgcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactatagaatacgaattc

SEQ ID NO: 10 - DNA, 8152 bp, Artificial Sequence
  mH5-pp65-GUS-pLW51(GUS) plasmid DNA sequence
gaattcgttggtggtcgccatggatggtgttattgtatactgtctaaacgcgttagtaaaacatggcgaggaaataaatcatataaaaaatgatttcatgattaaaccatgttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgccattcctgttattatatccataggaaaggatagagatgtttgtgaactattaatctcatctgataaagcgtgtgcgtgtatagagttaaattcatataaagtagccattcttcccatggatgtttccttttttaccaaaggaaatgcatcattgattattctcctgtttgatttctctatcgatgcggcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctgggtaccaggcgcgcatttcattttgtttttttctatgctataaatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggcggcccgctcgagaaaaattgaaaataaatacaaaggttcttgagggttgtgttaaattgaaagcgagaaataatcataaataagccaccaccgtttaaacagtcgacggtatcgataagcttgatatcgaattcctgcagcccgtacgcgcaggcagcatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgatttttatggcgcgccctgcagggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccagaaactagttaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 11 - DNA, 2709 bp, Artificial Sequence
  HCMV IEfusion gene
atggtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa

SEQ ID NO: 12 - DNA, 46 bp, Artificial Sequence
  Primer “a” for construction of the IEfusion gene
agctttgtttaaacgccaccaccatggtcaaacagattaaggttcg

SEQ ID NO: 13 - DNA, 60 bp, Artificial Sequence
  Primer “b” for construction of the IEfusion gene
ggcatgattgacagcctgggcgaggatgtcaccctggtcagccttgcttctagtcaccat

SEQ ID NO: 14 - DNA, 69 bp, Artificial Sequence
  Primer “c” for construction of the IEfusion gene
tgttagcgtgggcccggtgctactggaatcgataccggcatgattgacagcctgggcgaggatgtcacc

SEQ ID NO: 15 - DNA, 28 bp, Artificial Sequence
  Primer “d” for construction of the IEfusion gene
tagcaccgggcccacgctaacaacccac

SEQ ID NO: 16 - DNA, 39 bp, Artificial Sequence
  Primer “e” for construction of the IEfusion gene
ttggcgcgcctttattttactgagacttgttcctcaggt

SEQ ID NO: 17 - protein, 9 aa, Artificial Sequence
  carboxyl terminus of the 580 amino acid primary sequence of the IE2 protein [Swiss Prot #P19893]
GPEDQNTSC

SEQ ID NO: 18 - protein, 17 aa, Artificial Sequence
  E1/e4-IE2/e5 (IEfusion) gene
MVTRSKADQGDILAQAV

SEQ ID NO: 19 - DNA, 2709 bp, Artificial Sequence
  Synthetic polynucleotide IEfusion-VacO
atggtgaagcaaatcaaggtcagagtggacatggtaagacacagaattaaggaacacatgttgaagaagtatactcaaacagaggagaagttcaccggtgccttcaatatgatgggtggatgtctacagaacgctttggatatcttagataaggtacatgaaccattcgaagaaatgaagtgcattggattgacaatgcaatcaatgtatgagaactacatagtgccagaggataagcgtgaaatgtggatggcatgcatcaaggagttacatgatgtatccaaaggagcagccaacaagctaggtggtgctttgcaagcgaaggcaagagcgaagaaggatgaattgagacgaaagatgatgtacatgtgctatcgaaacatcgaattcttcactaagaactcagcgtttcctaagactaccaatggatgcagtcaagctatggctgcgcttcagaacttgcctcaatgtagtcctgatgaaatcatggcatatgcacagaagatcttcaagatcttagatgaggaaagagacaaggtattgactcatatcgatcacatattcatggatatactaacaacatgtgtagaaacgatgtgtaacgagtacaaggtaacttcggacgcttgtatgatgactatgtacggaggaatatctctacttagtgagttctgtcgagttctatgctgttacgtattagaagaaactagtgtaatgttagcgaagagaccattgatcactaagcctgaagtgatctcggttatgaagagacgaatagaggagatctgtatgaaggtgttcgcacaatacatcttaggagctgatcctctaagagtgtgtagtccatcggtagacgatttgagagctatagcggaggaatctgacgaggaagaggcaatagttgcatacacacttgctacagctggagtatccagttctgattctcttgtaagtcctccggagtcacctgtgccagcaaccataccgttgagtagtgtgattgtggctgagaactcggatcaggaagagtctgagcaatccgatgaagaagaggaggaaggagcacaagaggagagagaagatactgtctctgtgaagagtgaacctgtatctgaaatcgaggaagtagcacctgaggaagaggaggatggagccgaagaaccaacagcttcgggtggtaagtcaactcatccgatggtaaccagatctaaggcagaccagggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggagcatactatgcctgtgactcatcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcagtaa

SEQ ID NO: 20 - DNA, 2709 bp, Artificial Sequence
  Synthetic polynucleotide IEfusion-4nt
atggtcaaacagattaaggttcgagtggacatggtgcggcatagaatcaaggagcacatgctgaagaagtatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttaggaggtgcactgcaggctaaggcccgtgctaagaaggatgaacttaggagaaagatgatgtatatgtgctacaggaatatagagttctttaccaagaactcagccttccctaagaccaccaatggctgcagtcaggccatggcggcactgcagaacttgcctcagtgctctcctgatgagattatggcttatgcccagaagatatttaagatcttggatgaggagagagacaaggtgctcacgcacattgatcacatatttatggatatcctcactacatgtgtggaaacaatgtgtaatgagtacaaggtcactagtgacgcttgtatgatgaccatgtacggaggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctaggtgccgatcctctgagagtctgctctcctagtgtggatgacctacgggccatcgccgaggagtcagatgaggaagaggctattgtagcctacactttggccaccgctggtgtcagctcctctgattctctggtgtcacctccagagtcacctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgctccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 21 - protein, 902 aa, Artificial Sequence
  Synthetic polypeptide IEfusion-Vac0
MVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 22 - protein, 902 aa, Artificial Sequence; identical to SEQ ID NO: 21
  Synthetic polypeptide IEfusion

SEQ ID NO: 23 - protein, 902 aa, Artificial Sequence; identical to SEQ ID NO: 21
  Synthetic polypeptide IEfusion-4nt

SEQ ID NO: 24 - DNA, 1491 bp, Artificial Sequence
  Synthetic polynucleotide IE2-Vac0
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggagcatactatgcctgtgactcatcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcagtaa

SEQ ID NO: 25 - DNA, 1491 bp, Artificial Sequence
  Synthetic polynucleotide IE2
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa

SEQ ID NO: 26 - DNA, 1491 bp, Artificial Sequence
  Synthetic polynucleotide IE2-4nt
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 27 - DNA, 1491 bp, Artificial Sequence
  Synthetic polynucleotide IE2 4nt H363A
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacacatccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 28 - DNA, 1491 bp, Artificial Sequence
  Synthetic polynucleotide IE2 4nt H369A
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacagcaccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 29 - DNA, 1491 bp, Artificial Sequence
  Synthetic polynucleotide IE2 4nt H363A/H369A
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacagcaccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 30 - DNA, 1488 bp, Artificial Sequence
  Synthetic polynucleotide IE2 H363A
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 31 - DNA, 1488 bp, Artificial Sequence
  Synthetic polynucleotide IE2 H369A
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacagcaccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 32 - DNA, 1488 bp, Artificial Sequence
  Synthetic polynucleotide IE2 H363A/H369A
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacagcaccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 33 - DNA, 1488 bp, Artificial Sequence
  Synthetic polynucleotide IE2 VacO H363A
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggaggctactatgcctgtgactcatcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcag

SEQ ID NO: 34 - DNA, 1488 bp, Artificial Sequence
  Synthetic polynucleotide IE2 VacO H369A
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggagcatactatgcctgtgactgctcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcag

SEQ ID NO: 35 - DNA, 1488 bp, Artificial Sequence
  Synthetic polynucleotide IE2 VacO H363A/H369A
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggaggctactatgcctgtgactgctcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcag

SEQ ID NO: 36 - protein, 496 aa, Artificial Sequence
  Synthetic polypeptide IE2-VacO
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 37 - protein, 496 aa, Artificial Sequence; identical to SEQ ID NO: 36
  Synthetic polypeptide IE2

SEQ ID NO: 38 - protein, 496 aa, Artificial Sequence; identical to SEQ ID NO: 36
  Synthetic polypeptide IE2-4nt

SEQ ID NO: 39 - protein, 496 aa, Artificial Sequence
  Synthetic polypeptide IE2 H363A
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 40 - protein, 496 aa, Artificial Sequence
  Synthetic polypeptide IE2 H369A
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 41 - protein, 496 aa, Artificial Sequence
  Synthetic polypeptide IE2 H363A/H369A
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 42-43 - skipped (2 entries)

SEQ ID NO: 44 - DNA, 1224 bp, Artificial Sequence
  Synthetic polynucleotide IE1 VacO
atggtgaagcaaatcaaggtcagagtggacatggtaagacacagaattaaggaacacatgttgaagaagtatactcaaacagaggagaagttcaccggtgccttcaatatgatgggtggatgtctacagaacgctttggatatcttagataaggtacatgaaccattcgaagaaatgaagtgcattggattgacaatgcaatcaatgtatgagaactacatagtgccagaggataagcgtgaaatgtggatggcatgcatcaaggagttacatgatgtatccaaaggagcagccaacaagctaggtggtgctttgcaagcgaaggcaagagcgaagaaggatgaattgagacgaaagatgatgtacatgtgctatcgaaacatcgaattcttcactaagaactcagcgtttcctaagactaccaatggatgcagtcaagctatggctgcgcttcagaacttgcctcaatgtagtcctgatgaaatcatggcatatgcacagaagatcttcaagatcttagatgaggaaagagacaaggtattgactcatatcgatcacatattcatggatatactaacaacatgtgtagaaacgatgtgtaacgagtacaaggtaacttcggacgcttgtatgatgactatgtacggaggaatatctctacttagtgagttctgtcgagttctatgctgttacgtattagaagaaactagtgtaatgttagcgaagagaccattgatcactaagcctgaagtgatctcggttatgaagagacgaatagaggagatctgtatgaaggtgttcgcacaatacatcttaggagctgatcctctaagagtgtgtagtccatcggtagacgatttgagagctatagcggaggaatctgacgaggaagaggcaatagttgcatacacacttgctacagctggagtatccagttctgattctcttgtaagtcctccggagtcacctgtgccagcaaccataccgttgagtagtgtgattgtggctgagaactcggatcaggaagagtctgagcaatccgatgaagaagaggaggaaggagcacaagaggagagagaagatactgtctctgtgaagagtgaacctgtatctgaaatcgaggaagtagcacctgaggaagaggaggatggagccgaagaaccaacagcttcgggtggtaagtcaactcatccgatggtaaccagatctaaggcagaccagtaa

SEQ ID NO: 45 - DNA, 1194 bp, Artificial Sequence
  Synthetic polynucleotide IE1
atggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaa

SEQ ID NO: 46 - DNA, 1224 bp, Artificial Sequence
  Synthetic polynucleotide IE1-4nt
atggtcaaacagattaaggttcgagtggacatggtgcggcatagaatcaaggagcacatgctgaagaagtatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttaggaggtgcactgcaggctaaggcccgtgctaagaaggatgaacttaggagaaagatgatgtatatgtgctacaggaatatagagttctttaccaagaactcagccttccctaagaccaccaatggctgcagtcaggccatggcggcactgcagaacttgcctcagtgctctcctgatgagattatggcttatgcccagaagatatttaagatcttggatgaggagagagacaaggtgctcacgcacattgatcacatatttatggatatcctcactacatgtgtggaaacaatgtgtaatgagtacaaggtcactagtgacgcttgtatgatgaccatgtacggaggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctaggtgccgatcctctgagagtctgctctcctagtgtggatgacctacgggccatcgccgaggagtcagatgaggaagaggctattgtagcctacactttggccaccgctggtgtcagctcctctgattctctggtgtcacctccagagtcacctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgctccagaggaagaggaggatggtgctgaggaacccaccgcctctggaggcaagagcacccaccctatggtgactagaagcaaggctgaccagtaa